(19) United States
(12) Patent Application Publication
(10) Pub. No.: US 2019/0023743 A1
(43) Pub. Date: Jan. 24, 2019

(54) METHODS AND COMPOSITIONS FOR MODULATING DRUG-POLYMER ARCHITECTURE, PHARMACOKINETICS AND BIODISTRIBUTION

(71) Applicant: Duke University, Durham, NC (US)

(72) Inventors: Ashutosh Chilkoti, Durham, NC (US); John A. MacKay, Durham, NC (US); Matthew R. Dreher, Durham, NC (US)

(21) Appl. No.: 16/058,924

(22) Filed: Aug. 8, 2018

Related U.S. Application Data
(63) Continuation of application No. 13/942,037, filed on Jul. 15, 2013, now abandoned.
Application No. 13/942,037 is a continuation of application No. 12/743,990, filed on Feb. 22, 2011, now abandoned.
(60) Provisional application No. 61/003,871, filed on Nov. 20, 2007.

(51) Int. Cl. C07K 14/00; A61K 47/64; A61K 31/704

(52) U.S. Cl. CPC C07K 14/001; A61K 47/64; A61K 2300/00; A61K 31/704

(57) ABSTRACT

Drug-polymer chemotherapeutics are provided having improved therapeutic efficacy and reduced dose-limiting toxicity. Methods are also provided for modulating the architecture, pharmacokinetics and biodistribution of drug-polymers and for reducing the dependence of transition temperature on concentration for drug-polymers.

### PRIORITY

This application is a continuation of U.S. patent application Ser. No. 13/942,037, filed Jul. 15, 2013, which application is a continuation of U.S. patent application Ser. No. 12/743,990, filed Feb. 22, 2011, which is a national stage filing under 35 U.S.C. 371 of International Patent Application No. PCT/US2008/084159, filed Nov. 20, 2008, which claims the benefit of U.S. Provisional Application No. 61/003,871, filed Nov. 20, 2007, which are hereby incorporated by reference in their entirety.

### GOVERNMENT INTEREST

The presently disclosed subject matter was made with United States Government support under Grant Nos. 1 R01 EB007205 and R01 EB00188-01 awarded by NIH/NIBIB, and Grant No. F32CA123889 awarded by NIH/NCI. Accordingly, the United States Government has certain rights in the presently disclosed subject matter.

### TECHNICAL FIELD

The presently disclosed subject matter relates to methods for modulating the architecture of drug-polymers through selective placement of the drug molecule along the backbone of the polymer. The methods of the presently disclosed subject matter are useful for improving the toxicity, pharmacokinetics and biodistribution of polymer drugs and, in particular, for developing chemotherapeutic molecules with increased anti-tumor therapeutic efficacy and reduced toxicity.

### BACKGROUND

Conventional chemotherapeutics, including doxorubicin, have significant dose limiting toxicities. While chemotherapeutics are frequently successful at halting or reversing tumor progression, their use is hampered by toxicity within healthy tissues of the body. One approach to improve efficacy has been to chemically attach drug to high molecular weight polymers. Following intravenous administration, these polymers reduce drug accumulation in healthy tissues. Clearance of drug depends strongly upon molecular weight; therefore, a polymer drug conjugate of sufficient size is retained within the blood for long periods from hours to days. During this period, a significant fraction of the dose has the opportunity to flow through the tumor where it may accumulate. Such long circulating polymers passively accumulate via tumor-specific gaps in vascular walls. As a result, a host of clinical trials have been performed using high molecular weight polymers that divert drug away from healthy tissues and into tumors.

Accordingly, there is a need in the field for drug-polymers with improved pharmacokinetic and biodistribution properties to increase therapeutic efficacy and reduce toxicity.

### SUMMARY

In some embodiments, the presently disclosed subject matter provides compositions for diverting a drug molecule away from healthy tissues and directing the drug molecule to tumor cells, the compositions comprising a high molecular weight polymer having one or more drug molecules attached at one terminus of the polymer, wherein the drug-polymer assembles into micelles. In some embodiments, the high molecular weight polymer is a polypeptide and the drug molecules are attached through amino acid residues of the polypeptide. In some embodiments, the amino acid residues to which the drug molecules are attached are cysteine, lysine, glutamic acid and aspartic acid residues. In some embodiments, the drug molecules are doxorubicin. In some embodiments the high molecular weight polymer is Elastin Like Protein (ELP).

In some embodiments, the presently disclosed subject matter provides compositions for diverting a drug molecule away from healthy tissues and directing the drug molecule to tumor cells, the compositions comprising a high molecular weight polymer including an amino acid sequence X₁[(G)ₘX₂]ₙ (SEQ ID NO:1) wherein X₁ and X₂ are chemically modifiable amino acids (including but not limited to lysine, cysteine, glutamic acid and aspartic acid) and wherein m=0 to 10 and n=4 to 50. The amino acid sequence is located at either the N- or C-terminus; and one or more drug molecules are attached at either or both the residues, X₁ and X₂, of the amino acid sequence.

In some embodiments, the drug molecule is doxorubicin. In some embodiments, the amino acid sequence is C(GGC)₇ (SEQ ID NO:2) and is present at the C-terminus of the high molecular weight polymer. In some embodiments, the drug molecule is doxorubicin and is attached to one or more of the cysteine residues of the amino acid sequence. In some embodiments, the drug molecule is attached to an average of about 5 of the cysteine residues of the amino acid sequence: C(GGC)₇ (SEQ ID NO:2).

In some embodiments, the high molecular weight polymer is an Elastin Like Protein (ELP) having amino acid sequence: MSKGPG(XGVPG)₁₆₀WP, wherein X is V:A:G occurring in a ratio of 1:8:7 (SEQ ID NO:3). In some embodiments, the high molecular weight polymer is ELP (SEQ ID NO:3), the amino acid sequence is C(GGC)₇ (SEQ ID NO:2) and is present at the C-terminus of the ELP, the drug molecule is doxorubicin and the doxorubicin is attached to an average of about 5 of the cysteine residues of the amino acid sequence through a maleimide-hydrazone linking group.

In some embodiments, the presently disclosed subject matter provides compositions for diverting a drug molecule away from healthy tissues and directing the drug molecule to tumor cells, the composition comprising a high molecular weight polymer comprising an ELP amino acid sequence: MSKGPG(XGVPG)₁₆₀WP, wherein X is V:A:G:C occurring in a ratio of 1:7:7:1 (SEQ ID NO:4); and three or more drug molecules are attached to the cysteine residues of the ELP sequence. In some embodiments, the drug molecule is doxorubicin. In some embodiments, the drug molecule is attached to an average of about 5 of the cysteine residues.

In some embodiments, the composition for diverting a drug molecule away from healthy tissues and directing the drug molecule to tumor cells is prepared for administration to a vertebrate subject, or as a pharmaceutical formulation for administration to humans.

In some embodiments, the presently disclosed subject matter provides a method of treating a subject having cancer, the method comprising administering a composition comprising a high molecular weight polymer having one or more drug molecules attached at one terminus of the polymer, wherein the drug-polymer assembles into micelles.

In some embodiments, the presently disclosed subject matter provides a method of treating a subject having cancer, the method comprising administering a composition comprising a high molecular weight polymer comprising an amino acid sequence: X₁[(G)ₘX₂]ₙ (SEQ ID NO:1) at either the N- or C-terminus, and one or more drug molecules attached to a residue of the amino acid sequence.

In some embodiments, the presently disclosed subject matter provides a method for designing a drug-polymer chemotherapeutic having increased efficacy relative to the drug alone, the method comprising attaching one or more drug molecules at one terminus of a high molecular weight polymer, wherein the drug-polymer conjugate assembles into micelles.

In some embodiments, the presently disclosed subject matter provides a method for designing a drug-polymer chemotherapeutic having increased efficacy relative to the drug alone, the method comprising attaching one or more drug molecules at one terminus of a high molecular weight polymer comprising an amino acid sequence X₁[(G)ₘX₂]ₙ (SEQ ID NO:1) at either the N- or C-terminus, by linking one or more drug molecules to the cysteine residues of the amino acid sequence and wherein the drug-polymer assembles into micelles.

In some embodiments, the presently disclosed subject matter provides a method for designing a drug-polymer chemotherapeutic having reduced dose-limiting toxicity relative to the drug alone, the method comprising attaching one or more chemotherapeutic drug molecules at one terminus of a high molecular weight polymer, wherein the drug-polymer assembles into micelles.

In some embodiments, the presently disclosed subject matter provides a method for designing a drug-polymer chemotherapeutic having reduced dose-limiting toxicity relative to the drug alone, the method comprising placing an amino acid sequence X₁[(G)ₘX₂]ₙ (SEQ ID NO:1) at the N- or C-terminus of a high molecular weight polymer and linking one or more chemotherapeutic drug molecules to a residue of the amino acid sequence, wherein the drug-polymer assembles into micelles.

In some embodiments, the presently disclosed subject matter provides a method for designing a drug-polymer therapeutic having reduced dependence of transition temperature on concentration, the method comprising attaching one or more drug molecules at one terminus of a high molecular weight polymer, wherein the drug-polymer assembles into micelles.

In some embodiments, the presently disclosed subject matter provides a method for designing a drug-polymer therapeutic having reduced dependence of transition temperature on concentration, the method comprising placing an amino acid sequence X₁[(G)ₘX₂]ₙ (SEQ ID NO:1) at the N- or C-terminus of a high molecular weight polymer and linking one or more drug molecules to a residue of the amino acid sequence and wherein the drug-polymer assembles into micelles.

In some embodiments, the presently disclosed subject matter provides a method for modulating the pharmacokinetics and biodistribution of a drug-polymer, the method comprising attaching one or more drug molecules at one terminus of a high molecular weight polymer, wherein the drug-polymer assembles into micelles.

In some embodiments, the presently disclosed subject matter provides a method for modulating the pharmacokinetics and biodistribution of a drug-polymer, the method comprising placing an amino acid sequence X₁[(G)ₘX₂]ₙ (SEQ ID NO:1) at the N- or C-terminus of a high molecular weight polymer and linking one or more drug molecules to a residue of the amino acid sequence, wherein the drug-polymer assembles into micelles.

In some embodiments, the residue of the amino acid sequence is cysteine, the high molecular weight polymer is ELP (SEQ ID NO:3), the drug molecule is doxorubicin, the amino acid sequence is C(GGC)₇ (SEQ ID NO:2) and the drug molecule is linked through one or more cysteine residues of the amino acid sequence.

Accordingly, it is an object of the presently disclosed subject matter to provide methods and compositions for diverting a drug molecule away from healthy tissues and directing the drug molecule to tumor cells for the treatment of cancer. These and other objects are achieved in whole or in part by the presently disclosed subject matter.

Objects of the presently disclosed subject matter having been stated above, other objects and advantages will become apparent upon a review of the following descriptions, figures and examples.

### BRIEF DESCRIPTION OF THE DRAWINGS

FIGS. 1A-1B are schematic diagrams showing two different Elastin-Like Protein (“ELP”) architectures for carrying doxorubicin. FIG. 1A: Doxorubicin molecules (represented as triangles) are chemically attached to an ELP polymer. When the doxorubicin molecules self associate, they are surrounded by an ELP corona. Shown on the left, doxorubicin molecules are distributed equally along the ELP polymer, and stable unimeric molecules of ˜8 nm in radius are formed upon association of the doxorubicin molecules. Alternatively, multiple doxorubicin molecules can be attached to a C-terminal block of the ELP polymer, and multimeric micelles of ˜15 nm in radius are formed instead upon doxorubicin self-association. FIG. 1B: The approximate structure of a single ELP molecule after attachment with doxorubicin. Doxorubicin molecules are activated with a maleimide-hydrazone linkage that enables site-specific attachment to free sulphydryls on cysteine residues of the ELP. In this example, there are eight cysteine points of attachment on the ELP.

FIGS. 2A-2B show how ELP having a doxorubicin tail forms multimeric, micelle-like structures. FIG. 2A: Dynamic light scattering was used to determine the hydrodynamic radius of particles formed by the chemical species in FIG. 1B. FIG. 2B: Similar sized particles were confirmed using Freeze Fracture Transmission Electron microscopy.

FIG. 3 is a graph demonstrating the hydrodynamic radius for unimeric and micelle formulations of doxorubicin-ELP. Dynamic light scattering was used to determine the hydrodynamic radius for the unimeric and micelle formulations in PBS at 25° C. Error bars indicate the 95% confidence interval (n=3).

FIGS. 4A-4B are graphs showing transition temperature as a function of concentration for ELP and doxorubicin-ELP. A graph for micelles is shown in FIG. 4A and a graph for unimers is shown in FIG. 4B. The transition temperature, Tₜ, for these formulations was determined in PBS by measuring the turbidity at a 350 nm wavelength as a function of temperature. Each graph shows the Tₜ of parent ELP with and without attached doxorubicin. Micelle and unimer formulations have a similar drug loading capacity, i.e. ˜five doxorubicin molecules/ELP. The lines in each graph indicate the best fit linear regression to the equation: Tₜ=m Log₁₀ [C]+b.

FIG. 5 is a bar graph of the slopes of the best-fit lines for the dependence of transition temperature on the logarithm of the concentration of ELP with and without attached doxorubicin. Depicted in the bar graph are unmodified ELP2 (unimer), ELP2 modified with doxorubicin (micelle), ELP10PB (unimer) and ELP10PB with doxorubicin (unimer). The regression line was fit to the equation: Tₜ=m Log₁₀ [C]+b, and the slope m is represented in the bar graph. Error bars indicate the 95% confidence interval.

FIG. 6 is a graph showing the dependence on polymer architecture of doxorubicin pharmacokinetics in mouse plasma. For both unimeric and micelle ELP formulations, mice were dosed with 5 mg drug/kg body weight. Samples were taken using tail vein-puncture at 1, 15, 30, 60, 120, 240, 480, and 1440 minutes. Doxorubicin was extracted from heparin treated plasma in acidified isopropanol overnight and concentrations were determined using fluorescence calibration curves. Error bars indicate the 95% confidence interval.

FIG. 7 is a bar graph showing concentration of doxorubicin in mice tumors. The mice were treated with free doxorubicin, micelle doxorubicin-ELP, or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

FIG. 8 is a bar graph showing the concentration of doxorubicin in mouse heart tissue. The mice were treated with free doxorubicin, micelle doxorubicin-ELP, or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

FIG. 9 is a bar graph showing the concentration of doxorubicin in mouse liver tissue. The mice were treated with free doxorubicin, micelle doxorubicin-ELP, or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

FIG. 10 is a bar graph showing the concentration of doxorubicin in mouse kidney tissue. The mice were treated with free doxorubicin, micelle doxorubicin-ELP, or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

FIG. 11 is a graph showing the toxicity of doxorubicin as estimated by body weight loss. Animals dosed near the maximum tolerated amount of doxorubicin lose body weight, and weight loss 4 days post doxorubicin administration is used in this experiment as a gross indicator of toxicity. Balb/C mice bearing C26 colon carcinoma tumors were systemically administered PBS, free doxorubicin, micelle doxorubicin-ELP, or unimer doxorubicin-ELP at 0, 12.5, 25, and 6.3 mg drug/kg body weight respectively. At these doses, free drug and micelle drug were approximately equally toxic. Unimeric drug was more toxic than micelle drug even at ¼^{th} the total dose. PBS did not cause any weight loss. Error bars indicate the standard deviation (n=5).

FIG. 12 is a graph showing that mouse tumors are temporarily eliminated after treatment with micelle doxorubicin-ELP. Eight days after subcutaneous implantation of C26 colon carcinoma tumor cells, Balb/C mice were randomized and treated. The mice were systemically administered either a PBS control, 12.5 mg drug/kg body weight free doxorubicin or 25 mg drug/kg body weight micelle doxorubicin-ELP. At these doses, free doxorubicin and micelle doxorubicin-ELP were approximately equally toxic. The treatment groups were blinded during tumor measurement. Tumor volume was calculated according to: volume=π*length*width²/6. At day 8, the micelle doxorubicin-ELP treated animals had significantly smaller tumor volumes than either the PBS treated or free doxorubicin treated mice (Wilcoxin signed rank test). Error bars indicate the standard deviation of the mean.

FIG. 13 is a graph demonstrating that mice carrying tumors survive longer after treatment with micelle doxorubicin-ELP. Eight days after subcutaneous implantation of C26 colon carcinoma tumor cells, Balb/C mice were randomized and treated. The mice were systemically administered either a PBS control or does of approximately equal toxicity of free doxorubicin at 12.5 mg drug/kg body weight or 25 mg drug/kg body weight micelle doxorubicin-ELP. Mice were sacrificed after losing >15% of their body weight due to tumor burden. The treatment groups were blinded during measurement. While free doxorubicin did not significantly effect survival time, micelle doxorubicin-ELP resulted in a doubling of survival time (Kaplan Meier analysis).

### DETAILED DESCRIPTION

While chemotherapeutics are frequently successful at halting or reversing tumor progression, their use is hampered by toxicity within healthy tissues of the body. Accordingly, the presently disclosed subject matter provides compositions and methods for optimizing therapeutic agents for the treatment of cancer that have improved efficacy and reduced dose-limiting toxicity. The methods of the presently disclosed subject matter involve the selective placement of drug molecules at predetermined sites along the backbone of a high molecular weight polymer to divert the drug molecule away from healthy tissues and direct it to tumor cells. Polymers in which drug molecules are attached at the terminus form micelle structures, whereas polymers having the drug molecules attached throughout the length of the polymer remain as single, unimeric molecules in solution. The presently disclosed subject matter demonstrates that the drug-polymer micelle formation is better tolerated than the unimeric formation, enabling greater than 4-fold as much drug to be safely administered. In addition, the presently disclosed subject matter reveals that administration of the drug-polymer micelle form to tumor laden mice results in a significantly greater reduction in tumor volume relative to administration of unmodified free drug.

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a drug molecule” includes a plurality of such drug molecules, and so forth.

The term “about”, as used herein when referring to a measurable value such as an amount of weight, time, residues etc. is meant to encompass variations of, in some embodiments ±20% or ±10%, in some embodiments ±5%, in some embodiments ±1%, in some embodiments ±0.5%, and in some embodiments ±0.1%, from the specified amount, as such variations are appropriate to perform the disclosed methods.

The term “drug-polymer” as used herein refers to the attachment of any small molecule that is useful as a drug to a high molecular weight polymer. The attachment of the drug can be limited to one terminus of the polymer, or the drug can be attached throughout the length of the polymer. One or more drug molecules can be attached to the polymer. The “polymers” of the presently disclosed subject matter as used herein refer to any biocompatible material, composition or structure that comprises one or more polymers, which can be homopolymers, copolymers, or polymer blends. The term “biocompatible” as used herein refers to any material, composition or structure that has essentially no toxic or injurious impact on the living tissues or living systems which the material, composition or structure is in contact with and produces essentially no immunological response in such living tissues or living systems. Generally, the methods for testing the biocompatibility of a material, composition or structure are well known in the art. The polymers of the presently disclosed subject matter include, but are not limited to, naturally occurring, non-naturally occurring and synthetic polymers. For example, the polymers of the presently disclosed subject matter can be naturally occurring amino acid sequences and non-naturally occurring amino acid sequences (such as, e.g., recombinant sequences including fragments and variants of naturally occurring sequences). The polymers of the invention can range in molecular weight from about 10 kD to about 125 kD, from about 30 kD to about 100 kD and from about 50 kD to about 75 kD.

The term “effective amount” as used herein refers to any amount of drug-polymer that elicits the desired biological or medicinal response (e.g. reduction of tumor size) in a tissue, system, animal or human that is being sought by a researcher, veterinarian, medical doctor or other clinician. In some embodiments, the “effective amount” can refer to the amount of active drug-polymer that is sufficient for targeting a tumor in a subject.

As used herein, the term “modulation” refers to a change in the pharmacokinetic and/or biodistribution properties of a drug-polymer using the methods of the presently disclosed subject matter. For example, the pharmacokinetic and/or biodistribution properties of the drug-polymers of the presently disclosed subject matter are different than the same properties exhibited by the free drug. For example, the attachment of drug molecules at the terminus of a high molecular weight polymer of the presently disclosed subject matter versus attachment of the same drug throughout the length of the polymer results in a longer plasma half-life for the drug-polymer having drug attached at the terminus.

The term “subject” as used herein refers to any invertebrate or vertebrate species. The methods disclosed herein are particularly useful in the treatment of warm-blooded vertebrates. Thus, the presently disclosed subject matter concerns mammals and birds. More particularly, provided is the treatment of mammals such as humans, as well as those mammals of importance due to being endangered (such as Siberian tigers), of economic importance (animals raised on farms for consumption by humans), and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

As used herein, “treatment” or “treating” means any manner in which one or more of the symptoms of a disorder are ameliorated or otherwise beneficially altered. Thus, the terms “treating” or “treatment” of a disorder as used herein includes: reverting the disorder, i.e., causing regression of the disorder or its clinical symptoms wholly or partially; preventing the disorder, i.e. causing the clinical symptoms of the disorder not to develop in a subject that can be exposed to or predisposed to the disorder but does not yet experience or display symptoms of the disorder; inhibiting the disorder, i.e., arresting or reducing the development of the disorder or its clinical symptoms; attenuating the disorder, i.e., weakening or reducing the severity or duration of a disorder or its clinical symptoms; or relieving the disorder, i.e., causing regression of the disorder or its clinical symptoms. Further, amelioration of the symptoms of a particular disorder by administration of a particular composition refers to any lessening, whether permanent or temporary, lasting or transient that can be attributed to or associated with administration of the disclosed composition.

### II. Representative Embodiments

In some embodiments, the presently disclosed subject matter provides methods for optimization of therapeutic agents for the treatment of cancer by selectively placing drug molecules at predetermined sites along the backbone of a high molecular weight polymer to divert the drug away from healthy tissues and direct it to tumor cells. Conventional chemotherapeutic drug molecules generally have significant dose limiting toxicities. While chemotherapeutics are frequently successful at halting or reversing tumor progression, their use is hampered by toxicity within healthy tissues of the body.

This fact has produced a host of clinical trials using high molecular weight polymers that divert drug away from healthy tissues and into the tumor. One approach to improve efficacy of chemotherapeutics has been to chemically attach hydrophobic drug molecules to high molecular weight polymers. Following intravenous administration, these polymers reduce drug accumulation in healthy tissues. Clearance of drug depends strongly upon molecular weight; therefore, a drug-polymer conjugate of sufficient size is retained within the blood for long periods from hours to days. During this period, a significant fraction of the dose has the opportunity to flow through the tumor where it may accumulate. Such long circulating polymers passively accumulate via tumor-specific gaps in vascular walls. Subsequently, the ideal polymer will release active drug and then degrade into harmless components.

In some embodiments of the presently disclosed subject matter, the anti-tumor effect of existing chemotherapeutics is improved. Attachment of hydrophobic drug molecules at the terminus of a high molecular weight polymer can alter the structure of the drug-polymer conjugate from a unimeric form to a micelle form. In some embodiments of the presently disclosed subject matter, inducement of the micelle form by the foregoing method results in drug-polymer compositions that are better tolerated in animals and have superior antitumor activity. The compositions and methods of the presently disclosed subject matter are useful with a variety of polymers, proteins, and drugs to initiate the micelle formation.

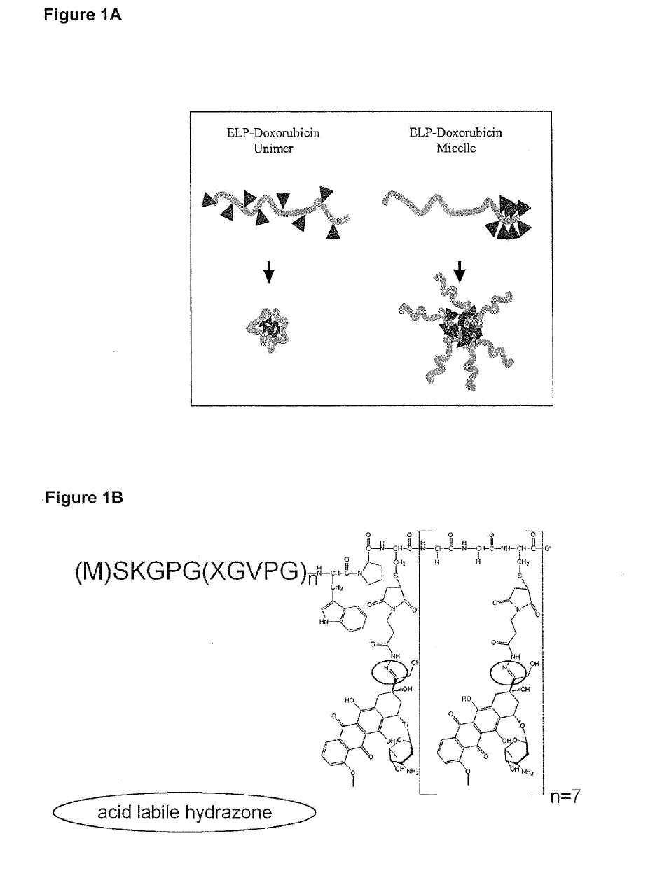

In some embodiments of the presently disclosed subject matter, Elastin-like-polypeptide (ELP) based polymers are well suited to meet the requirements for high molecular weight polymers having excellent properties for drug delivery approaches. For example, ELPs are a versatile set of biopolymers that can be easily produced and purified from E. coli with high efficiency, exact sequence specificity, and low polydispersity. Inspired from human elastin, ELP consists of repeats of Val-Pro-Gly-Xaa-Gly (SEQ ID NO:5), where the guest residue Xaa can be any amino acid except proline. In some embodiments, the presently disclosed subject matter describes an investigation of the architecture (FIG. 1) of a set of ELPs to which hydrophobic drug molecules have been attached at the terminus or along the polymer backbone (Table 1) (see Examples 1 & 2; Table I). The suitability of the resulting drug-polymers for treating animal tumor models is also described (see Examples 10-12).

In some embodiments, ELP have potential advantages over chemically synthesized polymers as drug delivery agents. First, because they are biosynthesized from a genetically encoded template, ELP can be made with precise molecular weight. Chemical synthesis of long linear polymers does not typically produce an exact length, but instead a range of lengths. Consequently, fractions containing both small and large polymers yield mixed pharmacokinetics and biodistribution. Second, ELP biosynthesis produces very complex amino acid sequences with nearly perfect reproducibility. This enables very precise selection of the location of drug attachment. Thus drug can be selectively placed on the corona, buried in the core, or dispersed equally throughout the polymer. Third, ELP can self assemble into multi-molecular micelles (see FIG. 1B) that can have excellent tumor accumulation and drug carrying properties. Due to their large diameter, multi-molecular micelles have different pharmacokinetics than smaller uni-molecular micelles. Fourth, because ELP are designed from native amino acid sequences found extensively in the human body they are biodegradable, biocompatible, and tolerated by the immune system. Fifth, ELP undergo an inverse phase transition temperature, Tₜ, above which they phase separate into large aggregates. By localized heating, additional ELP can be drawn into the tumor, which may be beneficial for increasing drug concentrations.

Accordingly, in some embodiments of the presently described subject matter, compositions are provided for diverting drug molecules away from healthy tissues and directing the drug molecules to tumor cells, the compositions comprising a high molecular weight polymer such as ELP to which one or more hydrophobic drug molecules are attached either along the length of the amino acid backbone (see FIG. 1A) or the hydrophobic drug molecules are attached at the end of the polymer (see FIG. 1B).

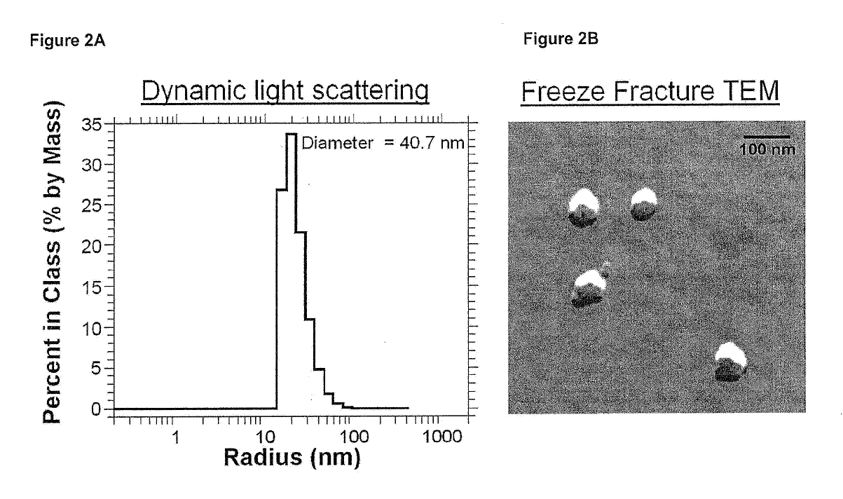

In some embodiments of the presently described subject matter, drug molecules are attached to the high molecular weight polymers through cysteine, lysine, glutamic acid or aspartic acid residues present in the polymer. In some embodiments, the cysteine, lysine, glutamic acid or aspartic acid residues are generally present throughout the length of the polymer. In some embodiments, the cysteine, lysine, glutamic acid or aspartic acid residues are clustered at the end of the polymer. In some embodiments of the presently described subject matter, drug molecules are attached to the cysteine residues of the high molecular weight polymer sequence using thiol reactive linkers. In some embodiments, the drug molecule is doxorubicin and it is attached to the polymer via cysteine-maleimide chemistry to a hydrazone activated doxorubicin[1] (see FIG. 2). In some embodiments of the presently described subject matter, drug molecules are attached to the lysine residues of the high molecular weight polymer sequence using NHS (N-hydroxysuccinimide) chemistry to modify the primary amine group present on these residues. In some embodiments of the presently described subject matter, drug molecules are attached to the glutamic acid or aspartic acid residues of the high molecular weight polymer sequence using EDC (1-Ethyl-3-[3-dimethylaminopropyl]carbodiimide Hydrochloride) chemistry to modify the carboxylic acid group present on these residues.

In some embodiments of the presently disclosed subject matter, the hydrophobic drug molecule is attached at the terminus of the high molecular weight polymer, and this configuration of hydrophobic drug induces the formation of micelles. In some embodiments, the high molecular weight polymer is a polypeptide. In some embodiments, the high molecular weight polymer is an ELP polypeptide. In some embodiments, the hydrophobic drug molecule is the chemotherapeutic agent, doxorubicin. In some embodiments, the average number of drug molecules attached to the polymer is about five (see, e.g. Table I).

In some embodiments, a peptide sequence comprising the sequence X₁[(G)ₘX₂]ₙ (SEQ ID NO:1) is appended to either the N or C-terminus of the polymer. In some embodiments, the compositions comprising a high molecular weight polymer include an amino acid sequence X₁[(G)ₘX₂]ₙ (SEQ ID NO:1) wherein X₁ and X₂ are chemically modifiable amino acids (including but not limited to lysine, cysteine, glutamic acid and aspartic acid) and wherein m=0 to 10 and n=4 to 50. The amino acid sequence is located at either the N- or C-terminus, and one or more drug molecules are attached at either or both the residues, X₁ and X₂, of the amino acid sequence. In some embodiments, the sequence C(GGC)₇ (SEQ ID NO:2) is appended to the polymer. In some embodiments, the sequence C(GGC)₇ (SEQ ID NO:2) is appended to the C-terminus of the polymer. In some embodiments, the polymer is a polypeptide. In some embodiments, the polymer is ELP. In some embodiments, the polymer is ELP (SEQ ID NO:3) and the sequence X₁[(G)ₘX₂]ₙ (SEQ ID NO:1) is appended to the C-terminus of the polymer. In some embodiments, the polymer is ELP (SEQ ID NO:3) and the sequence C(GGC)₇ (SEQ ID NO:2) is appended to the C-terminus of the polymer (see Example 1; Table I).

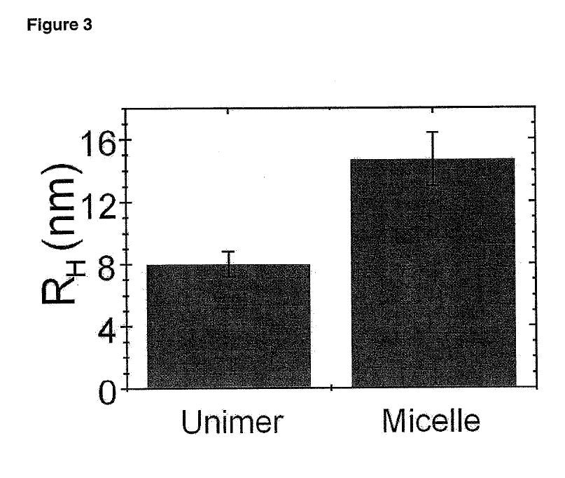

In some embodiments of the presently disclosed subject matter, a drug molecule such as doxorubicin is attached at the C-terminus of a high molecular weight polymer such as ELP (SEQ ID NO:3), and the resulting drug-polymer forms micelle structures under physiological salt and temperature conditions (see Example 2; FIG. 3). In some embodiments, the attachment points for a drug molecule such as doxorubicin are equally distributed along the backbone of the high molecular weight polymer such as ELP (SEQ ID NO:4), and the resulting drug-polymer is prevented from forming micelle structures under physiological salt and temperature conditions (see Example 2; FIG. 3). This molecule is here forth described as a unimer or unimeric. The sequence for a specific ELP (SEQ ID NO:4) polymer that can form a unimeric structure when drug molecules are attached is shown in Table I.

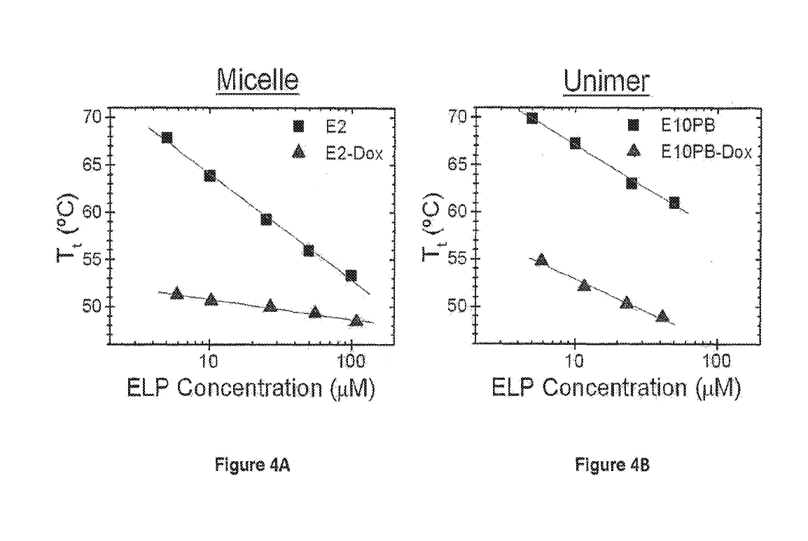

The attachment of drug molecules such as doxorubicin to a high molecular weight polymer such as ELP (SEQ ID NOs:3 and 4) decreases the transition temperature, Tₜ, for ELP for both micelle and unimeric ELP over a range of concentrations (see Example 3; FIG. 4). Attachment of hydrophobic drug molecules can significantly alter the apparent Tₜ of high molecular weight polymers.

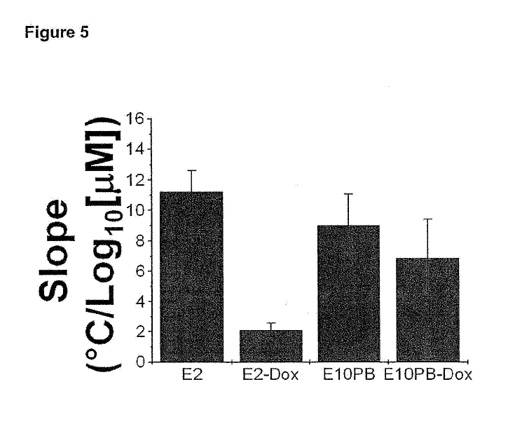

The formation of micelles by a drug-polymer of the presently disclosed subject matter can reduce the dependence of polymer transition temperature on concentration (see Example 4; FIG. 5). In some embodiments, the drug-polymer micelle compositions of the presently disclosed subject matter are useful for the development of thermally targeted drug-polymer therapeutics. Unimeric doxorubicin-ELP formulations demonstrate strong concentration dependence for Tₜ with an ˜10° C. increase in Tₜ, for a ten-fold change in concentration (see FIG. 5). This can result in a rapidly changing plasma Tₜ for any administered unimeric doxorubicin-ELP therapeutics. In contrast, a doxorubicin-ELP micelle formulation demonstrated only a 2° C. increase in Tₜ for every ten-fold change in concentration (see FIG. 5).

In some embodiments of the presently described subject matter, compositions are provided comprising a high molecular weight polymer having one or more hydrophobic drug molecules attached at a terminus of the polymer, which results in modulation of the biodistribution, toxicity, and anti-tumor therapeutic efficacy of the drug-polymer. Specific attachment of a drug molecule such as doxorubicin either along the backbone (see FIG. 1A) or at the end of the polymer (see FIG. 1B) enables the formation of different structures having differing drug delivery benefits. Attachment of the hydrophobic drug molecule at the terminus of the polymer results in formation of a micelle structure (see FIG. 1B), whereas placement of the drug along the length of the polymer results in the formation of a unimer structure (see FIG. 1A).

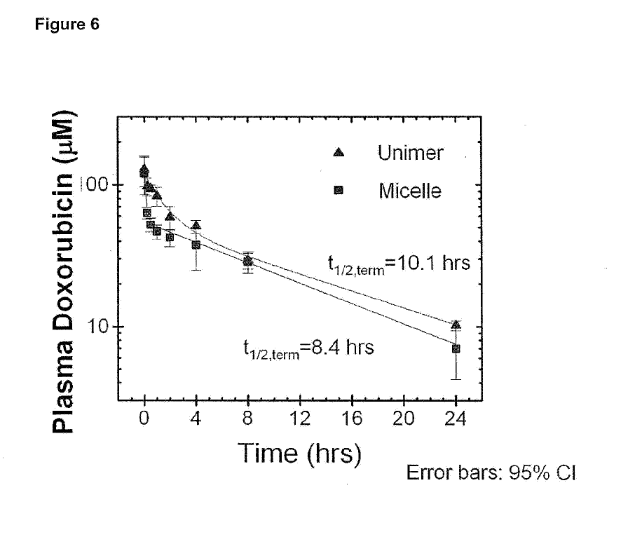

Micelle and unimeric drug-polymer compositions have significantly different plasma pharmacokinetics. While doxorubicin-ELP unimer and doxorubicin-ELP micelle demonstrated approximately the same terminal half-lives in mouse plasma (10.1 and 8.4 hrs, respectively), the compositions resulted in significantly different true half-lives (19 and 139 mins, respectively) (see Example 5; Table II, FIG. 6).

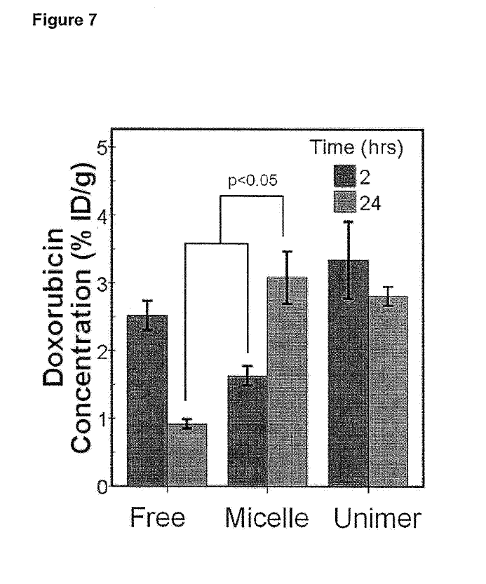

Both unimeric and micelle doxorubicin-ELP compositions accumulate to higher concentrations in mouse tumors than does free doxorubicin after 24 hours; however, unimeric doxorubicin-ELP achieves this concentration after only 2 hours (see Example 6; FIG. 7).

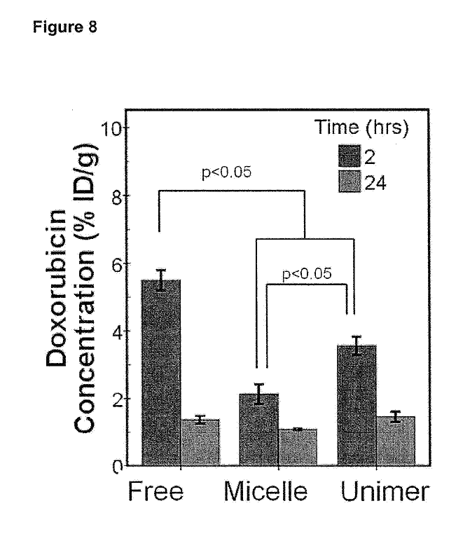

Doxorubicin-ELP micelle accumulates at lower concentrations in the heart than unimeric doxorubicin-ELP or free doxorubicin at short time periods (see Example 7; FIG. 8). This is beneficial because the heart is the site of dose-limiting toxicity for doxorubicin in humans.

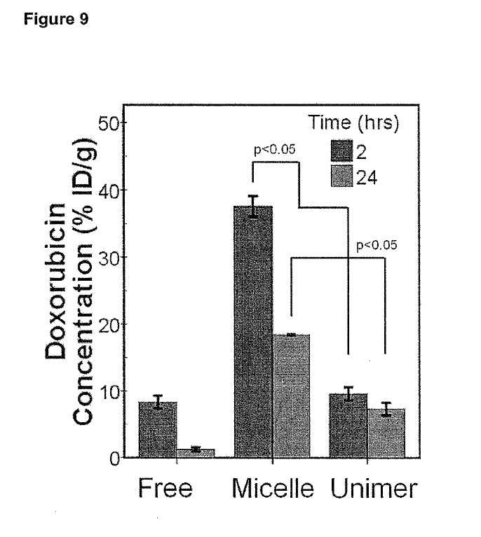

Doxorubicin-ELP micelles accumulate to higher concentrations in the liver than doxorubicin-ELP unimers or free doxorubicin. This is beneficial, because the liver is uniquely suited to degrade chemotherapeutics (see Example 8; FIG. 9).

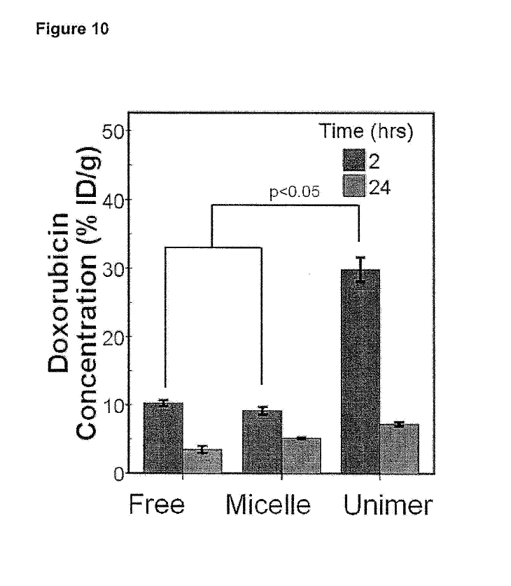

Doxorubicin-ELP unimers accumulate in the kidney after short times whereas doxorubicin-ELP micelles do not (see Example 9; FIG. 10). The smaller hydrodynamic radius for doxorubicin-ELP unimers appears to enable renal filtration and accumulation.

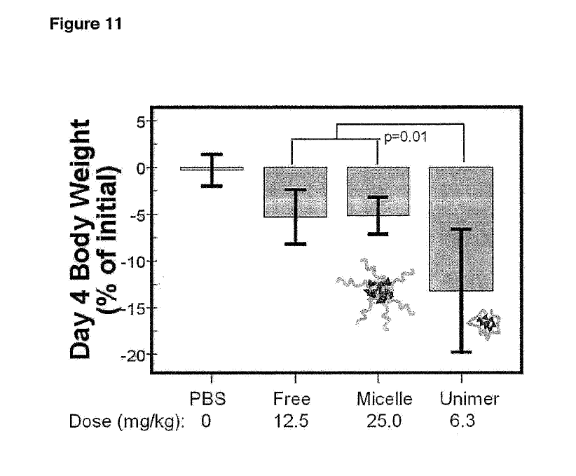

Doxorubicin-ELP micelles are better tolerated than free doxorubicin or doxorubicin-ELP unimers (see Example 10; FIG. 11). This is beneficial as it indicates that toxicity can be significantly influenced simply by moving the position of the drug molecule around the high molecular weight polymer backbone. This can have great clinical importance when it comes to designing polymer therapeutics to be well tolerated.

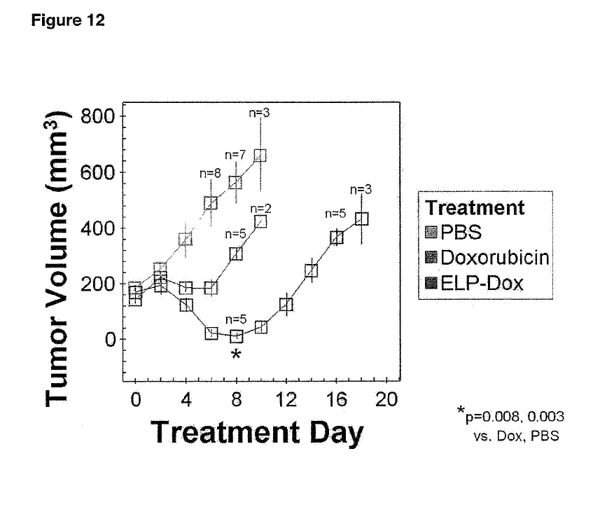

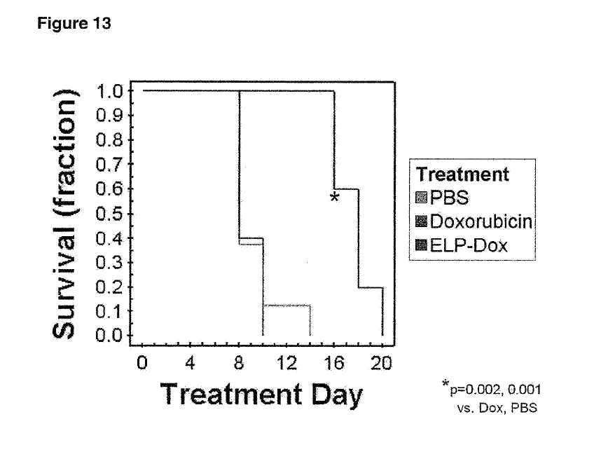

Doxorubicin-ELP micelles are more effective at reducing mouse tumor volume than an equally toxic dose of free doxorubicin (see Example 11; FIG. 12). Doxorubicin-ELP micelles improve survival of tumor laden mice compared to an equally toxic dose of free doxorubicin (see Example 12; FIG. 13).

Accordingly, in some embodiments of the presently described subject matter, a composition is provided for diverting a drug molecule away from healthy tissues and directing the drug molecule to tumor cells, the composition comprising a high molecular weight polymer having one or more drug molecules attached at one terminus of the polymer, wherein the drug-polymer assembles into micelles. In some embodiments, the composition is prepared for administration to a vertebrate subject, or as a pharmaceutical formulation for administration to humans.

In some embodiments of the presently described subject matter, a composition is provided for diverting a drug molecule away from healthy tissues and directing the drug molecule to tumor cells, the composition comprising a high molecular weight polymer comprising an amino acid sequence: X₁[(G)ₘX₂]ₙ (SEQ ID NO:1) at either the N- or C-terminus; and one or more drug molecules attached to a residue of the amino acid sequence.

In some embodiments, the drug molecule is doxorubicin. In some embodiments, the amino acid sequence is at the C-terminus of the high molecular weight polymer. In some embodiments, n is 7 (SEQ ID NO:2). In some embodiments, the drug molecule is attached to one or more of the cysteine residues of the amino acid sequence through a thiol reactive linking group. In some embodiments, the drug molecule is doxorubicin and the cysteine residue is attached through the linking group maleimide-hydrazone to the doxorubicin. In some embodiments, the drug molecule is attached to an average of about 5 of the cysteine residues of the amino acid sequence: C(GGC)₇ (SEQ ID NO:2).

In some embodiments, the high molecular weight polymer is an Elastin Like Protein (ELP) having amino acid sequence: MSKGPG(XGVPG)₁₆₀WP, wherein X is V:A:G occurring in a ratio of 1:8:7 (SEQ ID NO:3), the amino acid sequence is C(GGC)₇ (SEQ ID NO:2) and is present at the C-terminus of the ELP, the drug molecule is doxorubicin and the doxorubicin is attached to an average of about 5 of the cysteine residues of the amino acid sequence through a maleimide-hydrazone linking group.

In some embodiments of the presently disclosed subject matter, a composition is provided for diverting a drug molecule away from healthy tissues and directing the drug molecule to tumor cells, the composition comprising a high molecular weight polymer comprising an amino acid sequence MSKGPG(XGVPG)₁₆₀WP, wherein X is V:A:G:C occurring in a ratio of 1:7:7:1 (SEQ ID NO:4); and three or more drug molecules are attached to the cysteine residues of the amino acid sequence. In some embodiments, the drug molecule is doxorubicin. In some embodiments, the cysteine residue is attached through a linking group maleimide-hydrazone to the doxorubicin. In some embodiments, the drug molecule is attached to an average of about 5 of the cysteine residues.

In some embodiments of the presently disclosed subject matter, a method is provided for treating a subject having cancer, the method comprising administering a therapeutically effective amount of a composition comprising a high molecular weight polymer having one or more drug molecules attached at one terminus of the polymer, wherein the drug-polymer conjugate assembles into micelles. In some embodiments, the high molecular weight polymer comprises an amino acid sequence: X₁[(G)ₘX₂]ₙ (SEQ ID NO:1) at either the N- or C-terminus, and the one or more drug molecules are attached to a cysteine residue of the amino acid sequence. In some embodiments, the high molecular weight polymer is ELP (SEQ ID NO:3), the amino acid sequence is C(GGC)₇ (SEQ ID NO:2) and is present at the C-terminus of the ELP, the drug molecule is doxorubicin and the doxorubicin is attached to an average of about 5 of the cysteine residues of the amino acid sequence through a maleimide-hydrazone linking group.

In some embodiments of the presently disclosed subject matter, a method is provided for designing a drug-polymer chemotherapeutic having increased efficacy relative to the drug alone, the method comprising attaching one or more drug molecules at one terminus of a high molecular weight polymer, wherein the drug-polymer conjugate assembles into micelles. In some embodiments, the high molecular weight polymer comprises an amino acid sequence X₁[(G)ₘX₂]ₙ (SEQ ID NO:1) at the N- or C-terminus, and the one or more drug molecules are attached to the cysteine residues of the amino acid sequence. In some embodiments, the high molecular weight polymer is ELP (SEQ ID NO:3) and the drug molecule is doxorubicin.

In some embodiments of the presently disclosed subject matter, a method is provided for designing a drug-polymer chemotherapeutic having reduced dose-limiting toxicity relative to the drug alone, the method comprising attaching one or more drug molecules at one terminus of a high molecular weight polymer, wherein the drug-polymer conjugate assembles into micelles. In some embodiments, the high molecular weight polymer comprises an amino acid sequence X₁[(G)ₘX₂]ₙ (SEQ ID NO:1) at the N- or C-terminus and the one or more drug molecules are linked to the cysteine residues of the amino acid sequence. In some embodiments, the high molecular weight polymer is ELP (SEQ ID NO:3) and the drug molecule is doxorubicin.

In some embodiments of the presently disclosed subject matter, a method is provided for designing a drug-polymer therapeutic having reduced dependence of transition temperature on concentration, the method comprising attaching one or more drug molecules at one terminus of a high molecular weight polymer, wherein the drug-polymer conjugate assembles into micelles. In some embodiments, the high molecular weight polymer comprises an amino acid sequence X₁[(G)ₘX₂]ₙ (SEQ ID NO:1) at the N- or C-terminus and the one or more drug molecules are attached to the cysteine residues of the amino acid sequence. In some embodiments, the high molecular weight polymer is ELP (SEQ ID NO:3) and the drug molecule is doxorubicin.

In some embodiments of the presently disclosed subject matter, a method is provided for modulating the pharmacokinetics and biodistribution of a drug-polymer, the method comprising attaching one or more drug molecules at one terminus of a high molecular weight polymer, wherein the drug-polymer conjugate assembles into micelles. In some embodiments, the high molecular weight polymer comprises an amino acid sequence X₁[(G)ₘX₂]ₙ (SEQ ID NO:1) at the N- or C-terminus and the one or more drug molecules are linked to the cysteine residues of the amino acid sequence. In some embodiments, the high molecular weight polymer is ELP (SEQ ID NO:3) and the drug molecule is doxorubicin.

### REFERENCE

1. Furgeson, D. Y., Dreher, M. R., and Chilkoti, A. (2006). Structural optimization of a “smart” doxorubicin-polypeptide conjugate for thermally targeted delivery to solid tumors. J Control Release. 110: 362-369.

It will be understood that various details of the presently disclosed subject matter can be changed without departing from the scope of the presently disclosed subject matter. Furthermore, the foregoing description is for the purpose of illustration only, and not for the purpose of limitation.

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Generation of Doxorubicin-ELP Drug-Polymer

Approximately 5 doxorubicin molecules were attached to the end of an ELP polymer. The resulting drug-polymer was shown to form micelles (see Example 2 below). The ELP in Table I were produced in E. coli and attached via cysteine-maleimide chemistry to a hydrazone activated doxorubicin[1]. The specific C-terminal sequence used in this experiment was: ELP-Cys-Gly-Gly-Cys-Gly-Gly-CAs-Gly-Gly-Cys-Gly-Gly-Cys-Gly-Gly-Cys-Gly-Gly-Cys-Gly-Gly-Cys (SEQ ID NO:6; ELP=ELP2 in Table I).

TABLE I
Chemico-Physical Properties of Doxorubicin-ELP Conjugates.

| Architecture | Unimer | Micelle |
|---|---|---|
| ELP | ELP10PB | ELP2 |
| Sequence | | |
| Peptide | MSKGPG(XGVPG)₁₆₀WP | MSKGPG(XGVPG)₁₆₀WPC(GGC)₇ |
| Sequence | | |
| Guest | V:A:G:C [1:7:7:1] | V:A:G [1:8:7] |
| Residues (X) | | |
| Molecular. | 61.5 | 62.8 |
| weight (kD) | | |
| ¹Drug per | 4.8 ± 0.1 | 4.8 ± 1.3 |
| ELP | | |
| ²r_{H} (nm) | 8.0 ± 0.8 | 14.7 ± 1.7 |
| ³IC₅₀ (μM) | — | 2.0 ± 1.2 |
| ⁴pH 7.4 | −3 ± 4 | 1 ± 1 |
| release (%) | | |
| ⁵pH 5.0 | 99 ± 17 | 68 ± 3 |
| release, a (%) | | |
| ⁵pH 5.0 | 3.9 ± 1.5 | 4.9 ± 0.5 |
| t_{1/2} (hrs) | | |

¹ELP concentration determined by BCA assay against unmodified ELP in presence of 50 μM doxorubicin
²Particle radius determined by DLS at 25° C. in PBS. ± indicates 95% confidence interval (n = 3).
³Cytotoxicity measured in 96 well plates with 5,000 C26 cells per well incubated with dilutions of ELP-Dox and free dox following 3-day incubation. IC₅₀ free drug observed = 0.39 ± 0.19 μM. ± indicates standard deviation (n = 3).
⁴Average percentage of released free doxorubicin over 24 hours in pH 7.4 determined by HPLC. ± indicates 95% confidence interval.
⁵Nonlinear regression parameters for percentage of free doxorubicin released taken over 24 hours in pH 5.0 as determined by HPLC and fit to the equation F_{%,released} = a[1 − exp(−ln(2) t/t_{1/2})] where a is the maximum released and t_{1/2} is the first order half life of release. ± indicates 95% confidence interval.

After attachment with doxorubicin at the C-terminus as described above, the structure of a single ELP molecule was found to have the C-terminal chemistry shown in FIG. 1B. Specifically, doxorubicin was activated with a maleimide-hydrazone linkage that enabled site-specific attachment of the drug to the free sulphydryls on the cysteine residues at the C-terminal region of the ELP polymers (see FIG. 1B). In this conformation there are 8 cysteine points of attachment.

### Example 2

### ELP with Doxorubicin Tails Form Micelle Structures

The doxorubicin-ELP conjugate described in Example 1 and FIG. 1B was tested by two methods to determine if micelles are present under physiological salt and temperature. Dynamic light scattering was used to determine the hydrodynamic radius of particles formed by the chemical species in FIG. 1B. Similar sized particles were confirmed using Freeze Fracture Transmission Electron microscopy. The data in FIG. 2 show that ELP with doxorubicin tails form multimeric, micelle-like structures.

In contrast, the attachment of doxorubicin at equally distributed points along the ELP backbone prevents the formation of micelles. The specific sequence for this polymer is indicated in Table I (ELP10PB; SEQ ID NO:4). This molecule is referred to herein as a unimer or unimeric. FIG. 3 is a graph demonstrating the hydrodynamic radius for unimeric and micelle formulations of doxorubicin-ELP. Dynamic light scattering was used to determine the hydrodynamic radius for the unimeric and micelle formulations in PBS at 25° C. Error bars indicate the 95% confidence interval (n=3). Both the unimer and micelle formulations were found to have approximately 5 doxorubicin per molecule (Table I).

### Example 3

### Doxorubicin Attachment Decreases the Transition Temperature for ELP

Hydrophobic compounds can significantly alter the apparent transition temperature (Tₜ), of polymers, and this was shown to be case for both micelle and unimeric ELP over a range of concentrations (FIG. 4). FIGS. 4A-4B are graphs showing transition temperatures as a function of concentration for ELP and doxorubicin-ELP. The transition temperatures for these formulations were determined in PBS by measuring the turbidity at a 350 nm wavelength as a function of temperature. Each graph shows the Tₜ of parent ELP with and without attached doxorubicin (FIG. 4A is the micelle sequence, SEQ ID NO:3, and FIG. 4B is the unimer sequence, SEQ ID NO:4). Micelle and unimer formulations were determined to have a similar drug loading capacity, i.e. ˜5 doxorubicin/ELP. The lines in FIGS. 4A-4B indicate the best fit linear regression to the equation Tₜ=m Log₁₀ [C]+b.

### Example 4

### Micelle Formation Reduces Dependence of ELP Transition Temperature on Concentration

The slopes of the best-fit lines were plotted relating the dependence of transition temperature to the logarithm of the concentration of ELP (FIG. 5). Depicted in the bar graph of FIG. 5 are unmodified ELP2 (unimer), ELP2 modified with doxorubicin (micelle), ELP10PB (unimer), and ELP10PB with doxorubicin (unimer). The regression line was fit to the equation: Tₜ=m Log₁₀ [C]+b, and the slope m is plotted in FIG. 5. Error bars indicate the 95% confidence interval. For unimeric ELP formulations, a strong concentration dependence was observed on transition temperature. For example, there was about a 10° C. increase in Tₜ, for a ten-fold change in concentration of ELP. This result shows that the Tₜ for an ELP administered as a therapeutic would rapidly change in plasma. In contrast, the ELP micelle formulation showed only a 2° C. increase in Tₜ for every ten-fold change in concentration. The significantly decreased dependence of ELP transition temperature on concentration for micelle ELP is a useful effect for the development of thermally targeted ELP therapeutics.

### Example 5

### Micelle and Unimeric Drug-Polymers have Significantly Different Plasma Pharmacokinetics

While the data plotted in FIG. 6 show that ELP unimer and micelle forms have approximately the same terminal half-lives (10.1 and 8.4 hrs respectively), the true half-lives of the unimer and micelle forms are actually significantly different at 19 and 139 minutes, respectively (see Table II). To obtain the data for FIG. 6, mice were dosed with unimeric or micelle ELP formulations at 5 mg drug/kg body weight. Samples were taken using tail vein-puncture at 1, 15, 30, 60, 120, 240, 480, and 1440 minutes. Doxorubicin was extracted from heparin treated plasma in acidified isopropanol overnight and concentrations were determined using fluorescence calibration curves. Error bars indicate the 95% confidence interval. These data demonstrate how the pharmacokinetics of doxorubicin-ELP in mouse plasma depends on polymer architecture.

TABLE II
Comparative Two-compartment Pharmacokinetics
of Doxorubicin-ELP Conjugates

| | Treatment | |
|---|---|---|
| PK Parameters¹ | ELP2-Dox (n = 3) | ELP10PB-Dox (n = 4) |
| C₀ (uM) | 140 ± 36⁴ | 119 ± 13 |
| T₁ (min) | 5.6 ± 1.9 | 65.3 ± 46.7 |
| T₂ (hr) | 8.4 ± 0.7 | 10.1 ± 1.3 |
| α | 0.61 ± 0.11 | 0.55 ± 0.03 |
| T_{1/2} (min) | 19 ± 17³ | 139 ± 68² |
| AUC (nmol hr mL⁻¹) | 640 ± 68³ | 869 ± 47² |
| V₁ (mL g⁻¹) | 0.065 ± 0.021 | 0.073 ± 0.009 |
| Clearance (mL hr⁻¹ g⁻¹) | 0.0136 ± 0.0017 | 0.0099 ± 0.0005 |
| kₑ (hr⁻¹) | 0.22 ± 0.04 | 0.14 ± 0.02 |
| k₂₁ (hr⁻¹) | 3.08 ± 0.84 | 0.42 ± 0.20 |
| k₁₂ (hr⁻¹) | 4.90 ± 2.28 | 0.35 ± 0.18 |

¹Plasma concentrations profiles fit individually to ln[C(t)] = ln[C₀] + ln [α exp(−ln(2) t/T₁) + (1 − α)exp(−ln(2) t/T₂)]
²p < 0.05 by comparison to ELP2-Dox, Tukey HSD
³p < 0.05 by comparison to ELP10PB-Dox, Tukey HSD
⁴± indicates the observed standard deviation

### Example 6

### Higher Concentrations of Doxorubicin-ELP than Free Doxorubicin Accumulate in Tumors

The data in FIG. 7 show that for both unimeric and micelle doxorubicin-ELP, after 24 hours higher concentrations of the drug-polymer accumulate in tumors than for free doxorubicin. However, for unimeric doxorubicin-ELP this concentration is achieved after only 2 hours (FIG. 7). To determine the tumor concentration of doxorubicin, mice were treated with free doxorubicin, micelle doxorubicin-ELP, or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

### Example 7

### Doxorubicin-ELP Accumulation in Heart

Doxorubicin-ELP micelle accumulates at lower concentrations in the heart than unimeric doxorubicin-ELP or free doxorubicin at short time periods (FIG. 8). This is important because the heart is the site of dose-limiting toxicity for doxorubicin in humans. To determine heart concentrations of doxorubicin-ELP, mice were treated with free doxorubicin, micelle doxorubicin-ELP or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

### Example 8

### Doxorubicin-ELP Accumulation in Liver

Doxorubicin-ELP micelles accumulate at higher concentrations in the liver than doxorubicin-ELP unimers or free doxorubicin (FIG. 9). This is beneficial, because the liver is uniquely suited to degrade chemotherapeutics. To determine liver concentrations of doxorubicin-ELP, mice were treated with free doxorubicin, micelle doxorubicin-ELP or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

### Example 9

### Doxorubicin-ELP Accumulation in Kidney

Doxorubicin-ELP unimers accumulate in the kidney after short time periods, whereas doxorubicin-ELP micelles do not (FIG. 10). One possible explanation is the smaller hydrodynamic radius for ELP unimers allows for renal filtration and accumulation. To determine liver concentrations of doxorubicin-ELP, mice were treated with free doxorubicin, micelle doxorubicin-ELP or unimer doxorubicin-ELP formulations. Animals were dosed with 5 mg drug/kg body weight and tissues were obtained after 2 or 24 hours. Statistical comparison was performed using ANOVA followed by Tukey HSD post-hoc tests. The most relevant statistically significant comparisons have been indicated. Error bars indicate the standard error of the mean (n=4).

### Example 10

### Doxorubicin-ELP Micelles are Less Toxic than Free Doxorubicin or Doxorubicin-ELP Unimers

Doxorubicin-ELP micelles are better tolerated than free doxorubicin or doxorubicin-ELP unimers (FIG. 11). The toxicity of doxorubicin-ELP was estimated by body weight loss. Animals that were dosed near the maximum tolerated amount of free doxorubicin, micelle doxorubicin-ELP or unimer doxorubicin-ELP lost body weight, and the weight observed 4 days after the injection of the doxorubicin composition was taken as a gross indicator of toxicity. Balb/C mice bearing C26 colon carcinoma tumors were systemically administered either PBS as a control or free doxorubicin, micelle doxorubicin-ELP, or unimer doxorubicin-ELP at 12.5, 25, and 6.3 mg drug/kg body weight, respectively. At these doses, free doxorubicin and micelle doxorubicin-ELP were approximately equally toxic. Unimeric doxorubicin-ELP was more toxic than micelle doxorubicin-ELP even at ¼^{th} the total dose. The PBS control did not cause any weight loss. Error bars indicate the standard deviation (n=5). This is an important finding as it indicates that toxicity can be significantly influenced simply by moving the position of drug around the polymer backbone. This can have great clinical importance when it comes to designing polymer therapeutics to be well tolerated.

### Example 11

### Doxorubicin-ELP Micelles Show Greater Reductions in Tumor Mass than Free Doxorubicin at Equally Toxic Doses

The data in FIG. 12 show a greater reduction in tumor mass for doxorubicin-ELP micelles than free doxorubicin at an approximately equally toxic doses (FIG. 12). In fact, tumors are temporarily eliminated after treatment with micelle doxorubicin-ELP. The data shown in FIG. 12 were determined as follows: Eight days after subcutaneous implantation of C26 colon carcinoma tumor cells, Balb/C mice were randomized and treated. Mice were systemically administered a PBS control or approximately equally toxic doses of free doxorubicin or micelle doxorubicin-ELP at 12.5 and 25 mg drug/kg body weight, respectively. The treatment groups were blinded during tumor measurement. Tumor volume was measured according to the equation: volume=π*length*width²/6. At day 8, the micelle doxorubicin-ELP treated animals had significantly smaller tumor volumes than either the PBS treated or the free doxorubicin treated mice (Wilcoxin signed rank test). Error bars indicate the standard deviation of the mean.

### Example 12

### Mice Carrying Tumors Survive Longer after Treatment with Micelle Doxorubicin-ELP

Micelle doxorubicin-ELP improves survival as compared to an approximately equally toxic dose of free doxorubicin (FIG. 13). The data shown in FIG. 13 were determined as follows: Eight days after subcutaneous implantation of C26 colon carcinoma tumor cells, Balb/C mice were randomized and treated. Mice were systemically administered either a PBS control or approximately equally toxic doses of free doxorubicin or micelle doxorubicin-ELP at 12.5 and 25 mg drug/kg body weight, respectively. The mice were sacrificed after losing >15% of their body weight due to tumor burden. The treatment groups were blinded during measurement. Free doxorubicin did not have any significant effect on survival; however, micelle doxorubicin-ELP doubled the survival time significantly (Kaplan Meier analysis).

### REFERENCE

1. Furgeson, D. Y., Dreher, M. R., and Chilkoti, A. (2006). Structural optimization of a “smart” doxorubicin-polypeptide conjugate for thermally targeted delivery to solid tumors. J Control Release. 110: 362-369.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A composition for diverting a drug molecule away from healthy tissues and directing the drug molecule to tumor cells, the composition comprising:
(a) an Elastin Like Protein (ELP) having the ammo acid sequence MSKGPG(XGVPG)₁₀WP, wherein X is V:A:G occurring in a ratio of 1:8:7 (SEQ ID NO: 3), and further comprising the amino acid sequence C(GGC)₇ (SEQ ID NO:2) at either the N- or C-terminus; and
(b) one or more drug molecules attached to an average of about 5 of the cysteine residues of the amino acid sequence C(GGC)₇ (SEQ ID NO:2),
wherein the composition forms micelles.

2. The composition of claim 1, wherein the one or more drug molecules is doxorubicin.

3. The composition of claim 1, wherein the amino acid sequence C(GGC)₇ (SEQ ID NO:2) is at the C-terminus of the ELP.

4. The composition of claim 1, wherein the one or more drug molecules is attached to an average of about 5 of the cysteine residues of the amino acid sequence C(GGC)₇ (SEQ ID NO:2) through a thiol reactive linking group.

5. The composition of claim 4, wherein the one or more drug molecules is doxorubicin and the cysteine residue is attached through the linking group maleimide-hydrazone to the doxorubicin.

6. The composition of claim 2, the amino acid sequence C(GGC)₇ (SEQ ID NO:2) is present at the C-terminus of the ELP, and the doxorubicin is attached to an average of about 5 of the cysteine residues of the amino acid sequence C(GGC)₇ (SEQ ID NO:2) through a maleimide-hydrazone linking group.

7. The composition of claim 1, wherein the composition is prepared for administration to a vertebrate subject.

8. A method of treating a subject having cancer, the method comprising administering a therapeutically effective amount of a composition of claim 1 to the subject.

9. The composition of claim 7, wherein the composition is prepared as a pharmaceutical formulation for administration to humans.
